(19) United States
(12) Patent Application Publication
(10) Pub. No.: US 2018/0273897 A1
(43) Pub. Date: Sep. 27, 2018

(54) MODIFIED T-CELLS HAVING ANTI-FUGETACTIC PROPERTIES AND USES THEREOF

(71) Applicant: THE GENERAL HOSPITAL CORPORATION, Boston, MA (US)

(72) Inventors: Mark C. Poznansky, Newton Center, MA (US); Fabrizio Vianello, Padova (IT)

(21) Appl. No.: 15/760,774

(22) PCT Filed: Sep. 16, 2016
(86) PCT No.: PCT/US2016/052343

§ 371 Date: Mar. 16, 2018

Related U.S. Application Data
(60) Provisional application No. 62/220,857, filed on Sep. 18, 2015.
(60) Provisional application No. 62/220,927, filed on Sep. 18, 2015.
(60) Provisional application No. 62/303,365, filed on Mar. 3, 2016.
(60) Provisional application No. 62/303,368, filed on Mar. 3, 2016.

(51) Int. Cl. C12N 5/00; C12N 5/0783; A61K 35/17; A61K 31/395

(52) U.S. Cl. CPC C12N 5/0006; C12N 5/0636; C12N 2510/00; A61K 31/395; A61K 35/17

(57) ABSTRACT

This invention provides ex vivo methods for making modified immune cell, e.g., T cell, compositions having overall anti-fugetactic properties for the effective and efficient treatment of tumors or cancers in a patient, and compositions and use thereof.

### CROSS-REFERENCE TO RELATED APPLICATIONS

This application claims priority under 35 U.S.C. § 119(e) to U.S. Provisional Application Nos. 62/220,927, filed Sep. 18, 2015; 62/220,857, filed Sep. 18, 2015; 62/303,368; filed Mar. 3, 2016; and 62/303,365, filed Mar. 3, 2016; each of which is incorporated herein by reference in its entirety.

### BACKGROUND OF THE INVENTION

Cell movement in response to specific stimuli occurs in prokaryotes and eukaryotes. Cell movement seen in these organisms has been classified into three types: chemotaxis or the movement of cells along a gradient towards an increasing concentration of a chemical; negative chemotaxis which has been defined as the movement down a gradient of a chemical stimulus; and chemokinesis or the increased random movement of cells induced by a chemical agent.

Chemotaxis and chemokinesis occur in mammalian cells in response to the class of proteins, called chemokines. Additionally, chemorepellent, or fugetactic, activity has been observed in mammalian cells. For example, some tumor cells secrete concentrations of chemokines that are sufficient to repel immune cells from the site of a tumor, thereby reducing the immune system's ability to target and eradicate the tumor. Metastasizing cancer cells may use a similar mechanism to evade the immune system. Repulsion of T-cells, e.g. from a tumor expressing high levels of CXCL12 or interleukin 8 (IL-8), allows the tumor cells to evade immune control.

CXCR4 is a protein that in humans is encoded by the CXCR4 gene. CXCR4 is expressed by multiple normal cells as well as on tumors. CXCR4 is an alpha-chemokine receptor for stromal derived factor-1 (SDF-1, also known as CXCL12), a chemokine endowed with potent chemotactic activity for lymphocytes. As many as 85% of solid tumors and leukemias express CXCL12 at a level sufficient to have fugetactic effects, e.g., repulsion of immune cells from the tumor. Cancers that frequently express CXCL12 at such levels include, but are not limited to, prostate cancer, lung cancer, breast cancer, pancreatic cancer, ovarian cancer, gastric cancer, esophageal cancer, and leukemia.

Anti-fugetactic agents inhibit the fugetactic activity of tumor cells and allow the patient's immune system to target the tumor. Anti-fugetactic agents and the systemic delivery of anti-fugetactic agents are known in the art (see, for example, U.S. Patent Application Publication No. 2008/0300165, incorporated herein by reference in its entirety). However, the delivery of anti-fugetactic agents as heretofore described will likely result in a portion of the anti-fugetactic agent binding to the CXCR4 receptors on a tumor or other site, thus making the effective concentration of the anti-fugetactic agent that binds to immune cells, including T-cells, unpredictable.

Furthermore, immune cell therapy (i.e., infusion of autologous, allogenic, or immortalized immune cells into a patient) has shown that the infused immune cells may get “stuck” in particular tissues, leading to eradication of the infused immune cells before they are able to reach the target cancer cells.

Accordingly, there remains a need for treatments and compositions that target tumors to effectively and efficiently kill tumors and/or metastasizing cancer cells.

### SUMMARY OF THE INVENTION

Anti-fugetactic agents, such as AMD3100, associate with or bind to immune cells, e.g., T-cells, thereby blocking the fugetactic activity of chemokines with respect to the immune cells and allowing the immune cells to target a tumor or cancer cell. The association or binding can be by any suitable mechanism, including for example, via binding to CXCR4 receptors on the immune cells. Surprisingly, the anti-fugetactic property of these anti-fugetactic agents has been found to be concentration-dependent. In particular, it has been discovered that when an immune cell encounters too high a concentration of an anti-fugetactic agent, the anti-fugetactic effect is lost. The immune cell is thus prevented from effectively penetrating a tumor or homing in on metastasizing cancer cell.

CXCR4 receptors are found in multiple tissues as well as on tumors. Further, the T-cell population in the human body approaches or exceeds one trillion cells. While not wishing to be bound by theory, it is contemplated that the systemic delivery of an anti-fugetactic agent that targets a cell surface receptor expressed on immune cells, e.g. CXCR4, results in indiscriminate binding of that agent to receptors throughout the body. This binding dilutes the agent, potentially rendering it less efficient for the in vivo modification of enough immune cells to be anti-fugetactic and to efficiently and effectively eradicate tumors and/or cancer cells in a patient.

Based at least in part on the discoveries set forth above, it has been found that the binding of an anti-fugetactic agent to T-cells having CXCR4 receptors, ex vivo, provides an improved ability to control the amount of binding of the anti-fugetactic agent to the CXCR4 receptors on the T-cell to provide a modified cell population that, overall, retains the desired anti-fugetactic properties when administered to the patient. That is, the modified cell population is able to overcome the fugetactic effect of a tumor or cancer cell in order to effectively target the tumor or cell.

The immune cells having the anti-fugetactic agent bound to CXCR4 receptors on the cell surface are contemplated to have improved tumor penetration compared to cells that were not contacted with the anti-fugetactic agent prior to administration. In addition, the modified immune cells as described herein are contemplated to better target and penetrate tumors and cancer cells, and to avoid getting “stuck” in non-cancerous/non-target tissues.

Treatment of the patient with unbound anti-fugetactic agent prior to or concurrently with administration of the modified immune cells provides further improvements in anti-fugetactic response and tumor targeting of the immune cells. In particular, it is contemplated that the treatment with unbound anti-fugetactic agent will result in less competition for the anti-fugetactic agent bound to CXCR4 on the infused immune cells. That is, at least a subset of endogenous CXCR4 receptors encountered by the infused cells will be occupied by the anti-fugetactic agent and thus will not be available to compete away anti-fugetactic agent associated with the infused cells.

According to the present invention, such modified cell populations can be administered via any suitable method. In some embodiments, the modified cell population is administered locally to, or adjacent to, a tumor, tumor site(s) or cancer cells. Alternatively, the modified cell populations may be administered systemically, e.g., by intravenous infusion.

Similarly, the unbound anti-fugetactic agent can be administered via any suitable method, including locally or systemically.

In one aspect, the present disclosure relates to an ex vivo immune cell population comprising modified human immune cells, said immune cell population having an anti-fugetactic agent bound to individual immune cells. In one embodiment, the anti-fugetactic agent is bound to the cells through a receptor on the cell surface. In one embodiment, the receptor is CXCR4. In one embodiment, varying amounts of the anti-fugetactic agent are bound to individual immune cells. In one embodiment, at least a portion of the receptors on each cell are occupied by the agent.

In one embodiment, the immune cell population exhibits overall anti-fugetactic properties relative to a cancer when delivered to a patient in vivo. In one embodiment, the immune cell population is able to (has enhanced ability to) penetrate a tumor in vivo when delivered to a patient. In one embodiment, the immune cell has improved ability to target a tumor or cancer cell in vivo when delivered to a patient.

In one aspect, the present disclosure relates to a composition comprising a modified immune cell population comprising modified human immune cells, said immune cell population having an anti-fugetactic agent bound to individual immune cells. In one embodiment, the anti-fugetactic agent is bound to the cells through a CXCR4 receptor(s) on the cell surface. In one embodiment, varying amounts of the anti-fugetactic agent are bound to individual immune cells. In one embodiment, the immune cell population exhibits overall anti-fugetactic properties relative to a cancer when delivered to a patient in vivo. In one embodiment, the immune cell population is able to (has enhanced ability to) penetrate a tumor in vivo when delivered to a patient. In one embodiment, the immune cell has improved ability to target a tumor or cancer cell in vivo when delivered to a patient.

In a preferred embodiment, the immune cells are T-cells. In one embodiment, the T-cells are allogenic T-cells, autologous T-cells, or immortalized T-cells. In one embodiment, the T-cells are further modified to express a chimeric antigen receptor (also referred to as CAR-T cells).

In one embodiment, the CAR targets a cancer-associated antigen, for example, α-folate receptor, CAIX, CD19, CD20, CD30, CD33, CEA, EGP-2, erb-B2, erb-B 2,3,4, FBP, GD2, GD3, Her2/neu, IL-13R-a2, k-light chain, LeY, MAGE-Al, Mesothelin, and PSMA.

In one embodiment, the anti-fugetactic agent is selected from the group consisting of AMD3100 (mozobil/plerixafor) or derivative thereof, KRH-1636, T-20, T-22, T-140, TE-14011, T-14012, TN14003, TAK-779, AK602, SCH-351125, Tannic acid, NSC 651016, thalidomide, GF 109230X and an antibody that interferes with dimerization of a receptor for a fugetactic chemokine. In a preferred embodiment, the anti-fugetactic agent binds to CXCR4. Preferably, the anti-fugetactic agent is AMD3100.

In one embodiment, the composition further comprises a pharmaceutically acceptable excipient.

In one embodiment, the composition further comprises anti-fugetactic agent that is not bound to/associated with the immune cells.

In one embodiment, the immune cells are obtained from a patient having a cancer.

In one aspect, the present disclosure relates to a method of enhancing tumor penetration by immune cells in a patient having cancer, the method comprising administering to the patient an effective amount of a cell population or composition as described herein.

In one aspect, the present disclosure relates to a method of treating a patient having cancer, the method comprising:

a) providing immune cells;

b) modifying the immune cells by contacting the immune cells with an anti-fugetactic agent to provide modified immune cells as described herein; and

c) administering the modified immune cells to the patient so as to treat the cancer.

In some embodiments, providing the immune cells comprises extracting autologous immune cells from the patient.

In some embodiments, a therapeutically effective amount of the anti-fugetactic agent is administered systemically to the patient. In one embodiment, the therapeutically effective amount of the anti-fugetactic agent is administered prior to administration of the modified immune cells. In one embodiment, the therapeutically effective amount of the anti-fugetactic agent is administered concurrently with administration of the modified immune cells.

In some embodiments, the method further comprises modifying the immune cells to express a chimeric antigen receptor that is specific for the cancer.

In one aspect, the present disclosure relates to a method for making a modified immune cell composition, the method comprising (a) providing immune cells having CXCR4 receptors, and (b) contacting the immune cell population with an anti-fugetactic agent to provide a modified immune cell population as described herein.

In one embodiment, the immune cells are T-cells. In one embodiment, providing the T-cells includes extracting autologous immune cells having CXCR4 receptors from a patient having cancer to provide an immune cell population.

In one embodiment, the immune cells are contacted with said anti-fugetactic agent and stored for the subsequent administration to a patient. In one embodiment, the immune cells are contacted with the anti-fugetactic agent immediately prior to administration of the modified immune cell population to a patient.

One embodiment of the invention relates to an ex vivo method for making a modified autologous immune cell composition having overall anti-fugetactic properties, by (a) extracting autologous immune cells having CXCR4 receptors from a patient to provide an immune cell population, and (b) contacting the immune cell population with an anti-fugetactic agent to provide a modified immune cell population having anti-fugetactic properties for the effective and efficient treatment of tumors or cancers.

One embodiment of the invention relates to an ex vivo method for making a modified T-cell composition having overall anti-fugetactic properties, by (a) providing a T-cell population, and (b) contacting the T-cell population with an anti-fugetactic agent. to provide a modified T-cell population having anti-fugetactic properties for the effective and efficient treatment of tumors or cancers. In one embodiment, step (a) comprises extracting autologous T-cells from a patient to provide the T-cell population.

One embodiment of the invention relates to a method for treating tumors or cancers by the systemic administration of a modified immune cell composition as described herein to a patient in need thereof.

One embodiment of the invention relates to a method for treating tumors or cancers by the local administration of a modified immune cell composition as described herein to, or adjacent to, a tumor, tumor site(s), or cancer cells in a patient.

One embodiment of the invention relates to a method for treating tumors or cancers by the systemic administration of a modified T-cell composition as described herein to a patient in need thereof.

One embodiment of the invention relates to a method for treating tumors or cancers by the local administration of a modified T-cell composition according to the present invention to, or adjacent to, a tumor or site(s) or cancer cells in a patient in need thereof.

In one embodiment, the tumor is a solid tumor. In one embodiment, the tumor is a non-solid tumor. In one embodiment, the tumor is a leukemia.

### BRIEF DESCRIPTION OF THE FIGURES

FIG. 1 represents the bimodal chemotactic effect of increasing amounts of AMD3100 on human T cells.

FIG. 2 represents the bimodal fugetactic effect of increasing amounts of AMD3100 on human T cells.

### DETAILED DESCRIPTION OF THE INVENTION

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

### Definitions

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely examples and that equivalents of such are known in the art.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The terms “patient,” “subject,” “individual,” and the like are used interchangeably herein, and refer to any animal, or cells thereof whether in vitro or in situ, amenable to the methods described herein. In a preferred embodiment, the patient, subject, or individual is a mammal. In some embodiments, the mammal is a mouse, a rat, a guinea pig, a non-human primate, a dog, a cat, or a domesticated animal (e.g. horse, cow, pig, goat, sheep). In especially preferred embodiments, the patient, subject or individual is a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disease or disorder; (iii) slowing progression of the disease or disorder; and/or (iv) inhibiting, relieving, or slowing progression of one or more symptoms of the disease or disorder. For example, treatment of a cancer or tumor includes, but is not limited to, reduction in size of the tumor, elimination of the tumor and/or metastases thereof, remission of the cancer, inhibition of metastasis of the tumor, reduction or elimination of at least one symptom of the cancer, and the like.

The term “cancer” refers to a disease caused by an uncontrolled division of abnormal cells in a part or parts of the body. The term “tumor” refers to an abnormal mass of tissue. A tumor can be benign or malignant (cancerous).

The term “administering” or “administration” of an agent, drug, or an immune cell to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another. Preferably, administration is by intravenous administration or direct injection (e.g., to a tumor, near a tumor, or to a particular region of the body). For example, administration of the modified immune cells and/or anti-fugetactic agent can be by direct injection into the tumor. The modified immune cells and/or anti-fugetactic can alternatively be administered proximal to the tumor site or the modified immune cells and/or antifugetactic can be administered directly into a blood vessel associated with the tumor (e.g., via microcatheter injection into the blood vessels in, near, or feeding into the tumor).

It is also to be appreciated that the various modes of treatment or prevention of medical diseases and conditions as described are intended to mean “substantial,” which includes total but also less than total treatment or prevention, and wherein some biologically or medically relevant result is achieved.

The term “concurrent” administration refers to an administration of at least two active ingredients at the same time or substantially the same time, by the same or different routes.

The term “separate” administration refers to an administration of at least two active ingredients at the same time or substantially the same time by different routes.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

The term “simultaneous” therapeutic use refers to the administration of at least two active ingredients by the same route and at the same time or at substantially the same time.

The term “therapeutic” as used herein means a treatment and/or prophylaxis. A therapeutic effect is obtained by suppression, remission, or eradication of a disease state.

The term “therapeutically effective amount” or “effective amount” refers to an amount of the agent that, when administered, is sufficient to cause the desired effect. For example, an effective amount of an anti-fugetactic agent may be an amount sufficient to have an anti-fugetactic effect on a cancer cell or tumor (e.g. to attenuate a fugetactic effect from the tumor or cancer cell). The therapeutically effective amount of the agent will vary depending on the type of cancer being treated and its severity, as well as the age, weight, etc., of the patient to be treated. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

The term “kill” with respect to a cell/cell population is directed to include any type of manipulation that will lead to the death of that cell/cell population.

“Antibodies” as used herein include polyclonal, monoclonal, single chain, chimeric, humanized and human antibodies, prepared according to conventional methodology.

“Cytokine” is a generic term for non-antibody, soluble proteins which are released from one cell subpopulation and which act as intercellular mediators, for example, in the generation or regulation of an immune response. See Human Cytokines: Handbook for Basic & Clinical Research (Agrawal, et al. eds., Blackwell Scientific, Boston, Mass. 1991) (which is hereby incorporated by reference in its entirety for all purposes).

“CXCR4/CXCL12 antagonist” refers to a compound that antagonizes CXCL12 binding to CXCR4 or otherwise reduces the fugetactic activity of CXCL12.

By “fugetactic activity” or “fugetactic effect” it is meant the ability of an agent to repel (or chemorepel) a eukaryotic cell with migratory capacity (i.e., a cell that can move away from a repellant stimulus). The term also refers to the chemorepellent effect of a chemokine secreted by a cell, e.g. a tumor cell. Usually, the fugetactic effect is present in an area around the cell wherein the concentration of the chemokine is sufficient to provide the fugetactic effect. Some chemokines, including interleukin 8 and CXCL12, may exert fugetactic activity at high concentrations (e.g., over about 100 nM), whereas lower concentrations exhibit no fugetactic effect and may even be chemoattractant.

Accordingly, an agent with fugetactic activity is a “fugetactic agent.” Such activity can be detected using any of a variety of systems well known in the art (see, e.g., U.S. Pat. No. 5,514,555 and U.S. Patent Application Pub. No. 2008/0300165, each of which is incorporated by reference herein in its entirety). A preferred system for use herein is described in U.S. Pat. No. 6,448,054, which is incorporated herein by reference in its entirety.

The term “anti-fugetactic effect” refers to the effect of the anti-fugetactic agent to attenuate or eliminate the fugetactic effect of the chemokine.

The term “immune cells” as used herein are cells of hematopoietic origin that are involved in the specific recognition of antigens. Immune cells include antigen presenting cells (APCs), such as dendritic cells or macrophages, B cells, T cells, and the like.

The terms “T-cells” or “T lymphocytes”, as used herein, are a type of lymphocyte, i.e., a type of white blood cell, that plays a central role in cell-mediated immunity, and can be distinguished from other lymphocytes, such as B cells and natural killer cells (NK cells), by the presence of a T-cell receptor (TCR) on the cell surface. T-cells or T lymphocytes include several subsets of T-cells, each having a distinct function. Cytotoxic T-cells (CTLs) target and destroy virus-infected and tumor cells. Regulatory T-cells (Tregs) suppress immune responses of other cells and are recruited to many types of tumors; it is believed that Tregs may suppress the ability of the immune system to recognize and attack tumor cells. Other types of T-cells (including T helper cells, memory T-cells, and natural killer T-cells) are also involved in various aspects of tumor recognition and killing.

The term “T-cell receptor” or “TCR” is a complex of integral membrane proteins that participate in the activation of T-cells in response to an antigen. Stimulation of TCR is triggered by MHC (major histocompatibility complex) molecules on cells with the antigen. Engagement of the TCR initiates positive and negative cascades that ultimately result in cellular proliferation, differentiation, cytokine production, and/or activation-induced cell death. These signaling cascades regulate T-cell development, homeostasis, activation, acquisition of effector functions, and apoptosis.

The term “CD3” as used herein, also known as “cluster of differentiation 3” is a protein complex and is composed of four distinct chains. In mammals, the complex contains a CD3y chain, a CD3δ chain, and two CD3E chains. These chains associate with the T-cell receptor (TCR) and the ζ-chain to generate an activation signal in T lymphocytes. The TCR, ζ-chain, and CD3 molecules together comprise the TCR complex.

The term “autologous” or “autologous cells” as used herein refers to immune cells obtained from, and then administered to the same patient.

The term “allogenic” or “allogenic cells” as used herein refers to immune cells obtained from a subject other than the patient to whom they are administered. The terms “allogenic” and “allogeneic” are interchangeable herein.

The term “immortalized” or “immortalized cells” as used herein refers to immune cells that have been immortalized in vitro. That is, they are capable of growth and proliferation in in vitro cell culture.

The term “anti-cancer therapy” as used herein refers to traditional cancer treatments, including chemotherapy and radiotherapy, as well as immunotherapy and vaccine therapy.

As used herein “chimeric antigen receptors” or “CARs” refer to fusion proteins comprised of an antigen recognition moiety and T-cell activation domains. Eshhar et al., (1993) Proc. Natl. Acad. 90(2): 720-724. A CAR is an artificially constructed hybrid protein or polypeptide containing an antigen binding domain of an antibody (e.g., a single chain variable fragment (scFv)) linked to T-cell signaling or T-cell activation domains. CARs have the ability to redirect T-cell specificity and reactivity toward a selected target (i.e., a tumor cell) in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape. Moreover, when expressed in T-cells, CARs advantageously do not dimerize with endogenous T-cell receptor (TCI) alpha and beta chains.

### Anti-Fugetactic Agents

The anti-fugetactic agent may be any such agent known in the art. In one embodiment, the anti-fugetactic agent is an anti-fugetactic agent as described in U.S. Patent Application Publication No. 2008/0300165, which is hereby incorporated by reference in its entirety. In a preferred embodiment, the anti-fugetactic agent is selected from the group consisting of AMD3100 (mozobil/plerixafor; 1,1′[1,4-phenylenebis(methylene)]bis [1,4,8,11-tetraazacyclotetradecane]), KRH-1636, T-20, T-22, T-140, TE-14011, T-14012, TN14003, TAK-779, AK602, SCH-351125, Tannic acid, NSC 651016, thalidomide, GF 109230X, an antibody that interferes with dimerization of a fugetactic chemokine, and an antibody that interferes with dimerization of a receptor for a fugetactic chemokine. For example, the antibody may inhibit dimerization of CXCL12, IL-8, CXCR3, or CXCR4. In one embodiment, the anti-fugetactic agent is an antibody that interferes with binding of the chemokine to its receptor.

In a preferred embodiment, the anti-fugetactic agent is a CXCR4 antagonist. In an especially preferred embodiment, the anti-fugetactic agent is AMD3100.

In one embodiment, the anti-fugetactic agent is an AMD3100 derivative. AMD3100 derivatives include, but are not limited to, those found in U.S. Pat. Nos. 7,935,692 and 5,583,131 (USRE42152), each of which is incorporated herein by reference in its entirety.

Anti-fugetactic agents include any agents that specifically inhibit chemokine and/or chemokine receptor dimerization, thereby blocking the chemorepellent response to a fugetactic agent. Certain chemokines, including IL-8 and CXCL12 can also serve as chemorepellents at high concentrations (e.g., above 100 nM) where much of the chemokine exists as a dimer. Dimerization of the chemokine elicits a differential response in cells, causing dimerization of chemokine receptors, an activity which is interpreted as a chemorepellent signal. Blocking the chemorepellent effect of high concentrations of a chemokine secreted by a tumor can be accomplished, for example, by anti-fugetactic agents that inhibit chemokine dimer formation or chemokine receptor dimer formation. For example, antibodies that target and block chemokine receptor dimerization, e.g., by interfering with the dimerization domains or ligand binding, can be anti-fugetactic agents. Anti-fugetactic agents that act via other mechanisms of action, e.g., that reduce the amount of fugetactic cytokine secreted by the cells, inhibit dimerization, and/or inhibit binding of the chemokine to a target receptor, are also encompassed by the present invention. Where desired, this effect can be achieved without inhibiting the chemotactic action of the monomeric chemokine.

In other embodiments, the anti-fugetactic agent is a CXCR4 antagonist, CXCR3 antagonist, CXCR4/CXCL12 antagonist or selective PKC inhibitor.

The CXCR4 antagonist can be but is not limited to AMD3100, KRH-1636, T-20, T-22, T-140, TE-14011, T-14012, or TN14003, or an antibody that interferes with the dimerization of CXCR4. Additional CXCR4 antagonists are described, for example, in U.S. Patent Pub. No. 2014/0219952 and Debnath et al. Theranostics, 2013; 3(1): 47-75, each of which is incorporated herein by reference in its entirety, and include TG-0054 (burixafor), AMD3465, NIBR1816, AMD070, and derivatives thereof.

The CXCR3 antagonist can be but is not limited to TAK-779, AK602, or SCH-351125, or an antibody that interferes with the dimerization of CXCR3.

The CXCR4/CXCL12 antagonist can be but is not limited to Tannic acid, NSC 651016, or an antibody that interferes with the dimerization of CXCR4 and/or CXCL12.

The selective PKC inhibitor can be but is not limited to thalidomide or GF 109230X.

In a preferred embodiment, the anti-fugetactic agent is AMD3100 (plerixafor). AMD3100 is described in U.S. Pat. No. 5,583,131, which is incorporated by reference herein in its entirety.

In one embodiment, the anti-fugetactic agent is coupled with a molecule that allows targeting of a tumor or cancer. In one embodiment, the anti-fugetactic agent is coupled with (e.g., bound to) an antibody specific for the tumor to be targeted. In one embodiment, the anti-fugetactic agent is coupled to the molecule that allows targeting of the tumor or cancer.

### T-Cells

T cells are lymphocytes having T-cell receptor in the cell surface. T cells play a central role in cell-mediated immunity by tailoring the body's immune response to specific pathogens. T cells, especially modified T cells, have shown promise in reducing or eliminating tumors in clinical trials. Generally, such T cells are modified and/or undergo adoptive cell transfer (ACT). ACT and variants thereof are well known in the art. See, for example, U.S. Pat. Nos. 8,383,099 and 8,034,334, which are incorporated herein by reference in their entireties.

U.S. Patent App. Pub. Nos. 2014/0065096 and 2012/0321666, incorporated herein by reference in their entireties, describe methods and compositions for T cell or NK cell treatment of cancer. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005, each of which is incorporated herein by reference in its entirety.

In one embodiment, the T cells used in the compositions and methods herein are autologous T cells (i.e., derived from the patient). In one embodiment, the T cells used in the compositions and methods herein are non-autologous (heterologous; e.g. from a donor or cell line) T cells. In one embodiment, the T cell is a cell line derived from T cell(s) or cancerous/transformed T cell(s).

In one embodiment, the T cell used in the methods and compositions described herein is a genetically modified T cell. In one embodiment, the T cell is genetically modified to express a CAR on the surface of the T cell. In a preferred embodiment, the CAR is specific for the cancer being targeted by the method or composition. In one embodiment, the T cell is genetically modified to express a cell surface protein or cytokine. Non-limiting examples of genetically modified T cells are described in U.S. Pat. No. 8,906,682; PCT Patent Pub. Nos. WO 2013154760 and WO 2014055668; each of which is incorporated herein by reference in its entirety. See also, Wang and Riviere, Molecular Therapy—Oncolytics 3, Article number: 16015 (2016), which is incorporated herein by reference in its entirety.

In one embodiment, the T cell is a T cell line. Non-limiting examples of T cell lines include T-ALL cell lines, as described in U.S. Pat. No. 5,272,082, which is incorporated herein by reference in its entirety.

### Chimeric Antigen Receptors

Any CAR known to one of skill in the art now or in the future is encompassed by the present disclosure. In one embodiment, the CAR is specific for a tumor-specific antigen. Tumor-specific antigens can also be referred to as cancer-specific antigen. In one embodiment, the CAR is specific for a tumor-associated antigen. Tumor-associated antigens can also be referred to as cancer-associated antigen. A tumor-specific antigen is a protein or other molecule that is unique to cancer cells, while a tumor-associated antigen is an antigen that is highly correlated with certain tumor cells and typically are found at higher levels on a tumor cell as compared to on a normal cell. Tumor-specific antigens are described, by way of non-limiting example, in U.S. Pat. No. 8,399,645, U.S. Pat. No. 7,098,008; WO 1999/024566; WO 2000/020460; and WO 2011/163401, each of which is incorporated herein by reference in its entirety.

Examples of some known CARs are disclosure in Table 2. In one embodiment, the CAR targets a tumor-associated antigen, such as α-folate receptor, CAIX, CD19, CD20, CD30, CD33, CEA, EGP-2, erb-B2, erb-B 2,3,4, FBP, GD2, GD3, Her2/neu, IL-13R-a2, k-light chain, LeY, MAGE-Al, Mesothelin, or PSMA.

In some embodiments, the CAR recognizes an antigen associated with a specific cancer type, for example ovarian cancer, renal cell carcinoma, B-cell malignancies, Acute lymphoblastic leukemia (ALL), chronic lymphocytic leukemia (CLL), B-cell malignancies, refractory follicular lymphoma, mantle cell lymphoma, indolent B cell lyphoma, acute myeloid leukemia (AML), Hodgkin lymphoma, cervical carcinoma, breast cancer (including inflammatory breast cancer), colorectal cancer, prostate cancer, neuroblastoma, melanoma, rhabdornyosarcoma, medulloblastoma, adenocarcinomas, or tumor neovasculature.

TABLE 2
Chimeric Antigen Receptors

| | | | CARs |
|---|---|---|---|
| Target antigen | Associated malignancy | Receptor type | generation |
| α-Folate receptor | Ovarian cancer | ScFv-FcεRIγCAIX | First |
| CAIX | Renal cell carcinoma | ScFv-FcεRIγ | First |
| CAIX | Renal cell carcinoma | ScFv-FcεRIγ | Second |
| CD19 | B-cell malignancies | ScFv-CD3ζ (EBV) | First |
| CD19 | B-cell malignancies, CLL | ScFv-CD3ζ | First |
| CD19 | B-ALL | ScFv-CD28-CD3ζ | Second |
| CD19 | ALL | CD3ζ(EBV) | First |
| CD19 | ALL post-HSCT | ScFv-CD28-CD3ζ | Second |
| CD19 | Leukemia, lymphoma, CLL | ScFv-CD28-CD3ζ vs. | First and |
| | | CD3ζ | Second |
| CD19 | B-cell malignancies | ScFv-CD28-CD3ζ | Second |
| CD19 | B-cell malignancies post- | ScFv-CD28-CD3ζ | Second |
| | HSCT | | |
| CD19 | Refractory Follicular | ScFv-CD3ζ | First |
| | Lymphoma | | |
| CD19 | B-NHL | ScFv-CD3ζ | First |
| CD19 | B-lineage lymphoid | ScFv-CD28-CD3ζ | Second |
| | malignancies post-UCBT | | |
| CD19 | CLL, B-NHL | ScFv-CD28-CD3ζ | Second |
| CD19 | B-cell malignancies, CLL, B- | ScFv-CD28-CD3ζ | Second |
| | NHL | | |
| CD19 | ALL, lymphoma | ScFv-41BB-CD3ζ vs | First and |
| | | CD3ζ | Second |
| CD19 | ALL | ScFv-41BB-CD3ζ | Second |
| CD19 | B-cell malignancies | ScFv-CD3ζ (Influenza | First |
| | | MP-1) | |
| CD19 | B-cell malignancies | ScFv-CD3ζ (VZV) | First |
| CD20 | Lymphomas | ScFv-CD28-CD3ζ | Second |
| CD20 | B-cell malignancies | ScFv-CD4-CD3ζ | Second |
| CD20 | B-cell lymphomas | ScFv-CD3ζ | First |
| CD20 | Mantle cell lymphoma | ScFv-CD3ζ | First |
| CD20 | Mantle cell lymphoma, | CD3 ζ/CD137/CD28 | Third |
| | indolent B-NHL | | |
| CD20 | indolent B cell lymphomas | ScFv-CD28-CD3ζ | Second |
| CD20 | Indolent B cell lymphomas | ScFv-CD28-41BB- | Third |
| | | CD3ζ | |
| CD22 | B-cell malignancies | ScFV-CD4-CD3ζ | Second |
| CD30 | Lymphomas | ScFv-FcεRIγ | First |
| CD30 | Hodgkin lymphoma | ScFv-CD3ζ (EBV) | First |
| CD33 | AML | ScFv-CD28-CD3ζ | Second |
| CD33 | AML | ScFv-41BB-CD3ζ | Second |
| CD44v7/8 | Cervical carcinoma | ScFv-CD8-CD3ζ | Second |
| CEA | Breast cancer | ScFv-CD28-CD3ζ | Second |
| CEA | Colorectal cancer | ScFv-CD3ζ | First |
| CEA | Colorectal cancer | ScFv-FceRIγ | First |
| CEA | Colorectal cancer | ScFv-CD3ζ | First |
| CEA | Colorectal cancer | ScFv-CD28-CD3ζ | Second |
| CEA | Colorectal cancer | ScFv-CD28-CD3ζ | Second |
| EGP-2 | Multiple malignancies | scFv-CD3ζ | First |
| EGP-2 | Multiple malignancies | scFv-FcεRIγ | First |
| EGP-40 | Colorectal cancer | scFv-FcεRIγ | First |
| erb-B2 | Colorectal cancer | CD28/4-1BB-CD3ζ | Third |
| erb-B2 | Breast and others | ScFv-CD28-CD3ζ | Second |
| erb-B2 | Breast and others | ScFv-CD28-CD3ζ | Second |
| | | (Influenza) | |
| erb-B2 | Breast and others | ScFv-CD28mut-CD3ζ | Second |
| erb-B2 | Prostate cancer | ScFv-FcεRIγ | First |
| erb-B 2,3,4 | Breast and others | Heregulin-CD3ζ | Second |
| erb-B 2,3,4 | Breast and others | ScFv-CD3ζ | First |
| FBP | Ovarian cancer | ScFv-FcεRIγ | First |
| FBP | Ovarian cancer | ScFv-FcεRIγ | First |
| | | (alloantigen) | |
| Fetal | Rhabdomyosarcoma | ScFv-CD3ζ | First |
| acetylcholine | | | |
| receptor | | | |
| GD2 | Neuroblastoma | ScFv-CD28 | First |
| GD2 | Neuroblastoma | ScFv-CD3ζ | First |
| GD2 | Neuroblastoma | ScFv-CD3ζ | First |
| GD2 | Neuroblastoma | ScFv-CD28-OX40- | Third |
| | | CD3ζ | |
| GD2 | Neuroblastoma | ScFv-CD3ζ (VZV) | First |
| GD3 | Melanoma | ScFv-CD3ξ | First |
| GD3 | Melanoma | ScFv-CD3ξ | First |
| Her2/neu | Medulloblastoma | ScFv-CD3ξ | First |
| Her2/neu | Lung malignancy | ScFv-CD28-CD3ζ | Second |
| Her2/neu | Advanced osteosarcoma | ScFv-CD28-CD3ζ | Second |
| Her2/neu | Glioblastoma | ScFv-CD28-CD3ζ | Second |
| IL-13R-a2 | Glioma | IL-13-CD28-4-1BB- | Third |
| | | CD3ζ | |
| IL-13R-a2 | Glioblastoma | IL-13-CD3ζ | Second |
| IL-13R-a2 | Medulloblastoma | IL-13-CD3ζ | Second |
| KDR | Tumor neovasculature | ScFv-FcεRIγ | First |
| k-light chain | B-cell malignancies | ScFv-CD3ζ | First |
| k-light chain | (B-NHL, CLL) | ScFv-CD28-CD3ζ vs | Second |
| | | CD3ζ | |
| LeY | Carcinomas | ScFv-FcεRIγ | First |
| LeY | Epithelial derived tumors | ScFv-CD28-CD3ζ | Second |
| L1 cell adhesion | Neuroblastoma | ScFv-CD3ζ | First |
| molecule | | | |
| MAGE-A1 | Melanoma | ScFV-CD4-FcεRIγ | Second |
| MAGE-A1 | Melanoma | ScFV-CD28-FcεRIγ | Second |
| Mesothelin | Various tumors | ScFv-CD28-CD3ζ | Second |
| Mesothelin | Various tumors | ScFv-41BB-CD3ζ | Second |
| Mesothelin | Various tumors | ScFv-CD28-41BB- | Third |
| | | CD3ζ | |
| Murine CMV | Murine CMV | Ly49H-CD3ζ | Second |
| infected cells | | | |
| MUC1 | Breast, Ovary | ScFV-CD28-OX40- | Third |
| | | CD3ζ | |
| NKG2D ligands | Various tumors | NKG2D-CD3ζ | First |
| Oncofetal antigen | Various tumors | ScFV-CD3ζ | First |
| (h5T4) | | (vaccination) | |
| PSCA | Prostate carcinoma | ScFv-b2c-CD3ζ | Second |
| PSMA | Prostate/tumor vasculature | ScFv-CD3ζ | First |
| PSMA | Prostate/tumor vasculature | ScFv-CD28-CD3ζ | Second |
| PSMA | Prostate/tumor vasculature | ScFv-CD3ζ | First |
| TAA targeted by | Various tumors | FceRI-CD28-CD3ζ (+ | Third |
| mAb IgE | | a-TAA IgE mAb) | |
| TAG-72 | Adenocarcinomas | scFv-CD3ζ | First |
| VEGF-R2 | Tumor neovasculature | scFv-CD3ζ | First |

The immune cells can be genetically modified to express a desired CAR by any method known in the art. A vector containing a polynucleotide encoding a desired CAR can be readily introduced into the immune cells by physical, chemical, or biological means. Physical methods for introducing a polynucleotide into an immune cell include calcium phosphate precipitation, lipofection, particle bombardment, microinjection, electroporation, and the like. Methods for producing modified cells comprising vectors and/or exogenous nucleic acids are well-known in the art. See, for example, Sambrook et al. (2001, Molecular Cloning: A Laboratory Manual, Cold Spring Harbor Laboratory, New York). Biological methods for introducing a polynucleotide of interest into an immune cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Chemical means for introducing a polynucleotide into an immune cell include colloidal dispersion systems, such as macromolecule complexes, nanocapsules, microspheres, beads, and lipid-based systems including oil-in-water emulsions, micelles, mixed micelles, and liposomes.

### Modified Immune Cell Compositions

According to the present invention, a modified immune cell composition, preferably a modified T-cell composition, is prepared ex vivo (that is, outside of the body of a subject) by the methods described herein.

In one aspect, the present disclosure relates to an ex vivo T cell population comprising modified human T cells, said T cell population having an anti-fugetactic agent bound to individual T cells. In one embodiment, the anti-fugetactic agent is bound to the cells through a receptor on the cell surface. In one embodiment, the receptor is CXCR4. In one embodiment, varying amounts of the anti-fugetactic agent are bound to individual T cells. In one embodiment, at least a portion of the receptors on each cell are occupied by the agent. In one embodiment, the anti-fugetactic agent is bound to individual T cells.

In one embodiment, “at least a portion of the receptors” refers to at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of a particular type of receptors (e.g., CXCR4 receptors) are occupied by the agent.

In some embodiments, autologous immune cells for use in making the compositions described herein are extracted or otherwise isolated from blood, bone marrow, or other immune cell-containing organs of a patient having a cancerous tumor or other cancer, according to methods known in the art. For example, such methods include, but are not intended to be limited to, apheresis techniques, specifically leukapheresis. Additionally, commercially available kits may be utilized for the extraction of T-cells, such as with EasySep™ Human T Cell Isolation Kit available from STEMCELL™ Technologies, Inc., British Columbia, Canada.

In some embodiments, allogenic immune cells are extracted or otherwise isolated from blood, bone marrow, or other immune cell-containing organs of a subject other than the patient having the cancer to be treated. In some embodiments, the immune cells are expanded (i.e., the number of cells is increased, for example by growing the cells in culture).

In some embodiments, an immune cell line is provided.

In some embodiments, the immune cells are genetically modified. The immune cells may be genetically modified before or after expansion.

The immune cells are then contacted, mixed or otherwise combined with a predetermined amount of an anti-fugetactic agent as described herein, preferably AMD3100, under conditions such that the immune cell population has overall anti-fugetactic properties. For example, the conditions may allow the anti-fugetactic agent to bind to at least a subset of CXCR4 receptors on the surface of individual cells in the population. As would be understood by one skilled in the art, the amount of the anti-fugetactic agent can be determined, for example, as described in U.S. Patent Application Publication No. 2008/0300165, which is incorporated herein by reference in its entirety.

The immune cells are contacted with the anti-fugetactic agent to form a modified immune cell population or composition having anti-fugetactic properties (e.g., having an improved ability to target and/or penetrate a tumor), which can then be stored under conditions known in the art for blood products for the subsequent administration to a patient having cancer. In one embodiment, the immune cells are stored (and optionally extracted) under conditions known in the art for blood products, and then contacted with the anti-fugetactic agent immediately prior to administration of the modified immune cell population or composition to the patient. In another embodiment, the immune cells are contacted with the anti-fugetactic agent (and optionally extracted) immediately prior to administration of the modified immune cell population or composition to the patient.

### Anti-Cancer Therapy

In some embodiments, at least one additional anti-cancer therapy is administered in combination with the modified immune cells. The anti-cancer therapy may be any treatment which is used to treat cancer, including but not limited to, chemotherapy, radiation (e.g., proton beam therapy, brachytherapy, external beam therapy, etc.), immunotherapy, vaccine therapy, and the like.

In some embodiments, the anti-cancer therapy is administered prior to administration of the modified immune cells. In some embodiments, the anti-cancer therapy is administered after administration of the modified immune cells. In some embodiments, the anti-cancer therapy is administered concurrently with administration of the modified immune cells.

In some embodiments, the anti-cancer therapy is administered in the same composition as the modified immune cells. In some embodiments, the anti-cancer therapy and modified immune cells are administered in different compositions.

In some embodiments, administration of the anti-cancer therapy and the modified immune cells is alternated until a desired therapeutic outcome is reached.

### Dose and Administration

The modified immune cell compositions, as described herein, are administered in vivo to a patient in effective amounts. The effective amount will depend upon the mode of administration, the particular condition being treated and the desired outcome. It will also depend upon the stage of the condition, the age and physical condition of the subject, the nature of concurrent therapy, if any, and like factors well known to the medical practitioner. For therapeutic applications, it is that amount sufficient to achieve a medically desirable result.

The amount of the modified immune cell composition to be administered to the patient will depend, inter alia, on the type of immune cell that is used. Doses of autologous, allogenic, and/or immortalized T-cells are known in the art and can be determined by a qualified physician. In some embodiments, a reduced amount of cells may be used compared to a standard dose of immune cells that were not modified as described herein. Without being bound by theory, it is contemplated that improved targeting/penetration of the cells to the tumor will result in fewer total cells being required for treatment.

Generally, the dose of the modified immune cell composition of the present invention is from about 5 mg/kg body weight per day to about 50 mg/kg per day of the anti-fugetactic agent, inclusive of all values and ranges there between, including endpoints. In one embodiment, the dose is from about 10 mg/kg to about 50 mg/kg per day. In one embodiment, the dose is from about 10 mg/kg to about 40 mg/kg per day. In one embodiment, the dose is from about 10 mg/kg to about 30 mg/kg per day. In a preferred embodiment, the dose is from about 10 mg/kg to about 20 mg/kg per day. In one embodiment, the dose does not exceed about 50 mg per day.

Where an anti-fugetactic agent is administered in conjunction with the immune cells, the dose of the anti-fugetactic agent may be from about 5 mg/kg body weight per day to about 50 mg/kg per day, inclusive of all values and ranges there between, including endpoints. In one embodiment, the dose is from about 10 mg/kg to about 50 mg/kg per day. In one embodiment, the dose is from about 10 mg/kg to about 40 mg/kg per day. In one embodiment, the dose is from about 10 mg/kg to about 30 mg/kg per day. In a preferred embodiment, the dose is from about 10 mg/kg to about 20 mg/kg per day. In one embodiment, the dose does not exceed about 50 mg/kg per day.

In one embodiment, the dose of the modified immune cell composition and/or unbound anti-fugetactic agent is from about 50 mg/kg per week to about 350 mg/kg per week of the anti-fugetactic agent, inclusive of all values and ranges there between, including endpoints. In one embodiment, the dose is about 50 mg/kg per week. In one embodiment, the dose is about 60 mg/kg per week. In one embodiment, the dose is about 70 mg/kg per week. In one embodiment, the dose is about 80 mg/kg per week. In one embodiment, the dose is about 90 mg/kg per week. In one embodiment, the dose is about 100 mg/kg per week. In one embodiment, the dose is about 110 mg/kg per week. In one embodiment, the dose is about 120 mg/kg per week. In one embodiment, the dose is about 130 mg/kg per week. In one embodiment, the dose is about 140 mg/kg per week. In one embodiment, the dose is about 150 mg/kg per week. In one embodiment, the dose is about 160 mg/kg per week. In one embodiment, the dose is about 170 mg/kg per week. In one embodiment, the dose is about 180 mg/kg per week. In one embodiment, the dose is about 190 mg/kg per week. In one embodiment, the dose is about 200 mg/kg per week. In one embodiment, the dose is about 210 mg/kg per week. In one embodiment, the dose is about 220 mg/kg per week. In one embodiment, the dose is about 230 mg/kg per week. In one embodiment, the dose is about 240 mg/kg per week. In one embodiment, the dose is about 250 mg/kg per week. In one embodiment, the dose is about 260 mg/kg per week. In one embodiment, the dose is about 270 mg/kg per week. In one embodiment, the dose is about 280 mg/kg per week. In one embodiment, the dose is about 290 mg/kg per week. In one embodiment, the dose is about 300 mg/kg per week. In one embodiment, the dose is about 310 mg/kg per week. In one embodiment, the dose is about 320 mg/kg per week. In one embodiment, the dose is about 330 mg/kg per week. In one embodiment, the dose is about 340 mg/kg per week. In one embodiment, the dose is about 350 mg/kg per week.

In one aspect of the invention, administration of the modified immune cell composition and/or unbound anti-fugetactic agent is pulsatile for a period of time sufficient to have an anti-fugetactic effect (e.g. to attenuate the fugetactic effect of the tumor cell). In one embodiment, an amount of modified immune cell composition and/or unbound anti-fugetactic agent is administered every 1 hour to every 24 hours, for example every 1 hour, 2 hours, 3 hours, 4 hours, 5 hours, 6 hours, 7 hours, 8 hours, 9 hours, 10 hours, 11 hours, 12 hours, 13 hours, 14 hours 15 hours, 16 hours, 17 hours, 18 hours, 19 hours, 20 hours, 21 hours, 22 hours, 23 hours, or 24 hours. In one embodiment, an amount of modified immune cell composition and/or unbound anti-fugetactic agent is administered every 1 day, 2 days, 3 days, 4 days, 5 days, 6 days, 7 days, 8 days, 9 days, or 10 days.

A variety of administration routes are available. The methods of the invention, generally speaking, may be practiced using any mode of administration that is medically acceptable, meaning any mode that produces effective levels of the active compounds without causing clinically unacceptable adverse effects.

The modified immune cell composition and/or unbound anti-fugetactic agent can be administered in combination with at least one anti-cancer therapy/agent. “In combination” refers to any combination, including sequential or simultaneous administration. In one embodiment, the modified immune cell composition and/or unbound anti-fugetactic agent is administered separately from the anti-cancer therapy/agent. In one embodiment, the modified immune cell composition and/or unbound anti-fugetactic agent is administered in a single composition with the anti-cancer agent(s).

In one embodiment, the modified immune cell composition and/or unbound anti-fugetactic agent and/or anti-cancer agent is administered intravenously, subcutaneously, orally, or intraperitoneally. In an embodiment, the modified immune cell composition and/or unbound anti-fugetactic agent and/or anti-cancer agent is administered proximal to (e.g., near or within the same body cavity as) the tumor. In one embodiment, the modified immune cell composition and/or unbound anti-fugetactic agent and/or the at least one additional anti-cancer agent are administered directly to the tumor site. In one embodiment, the modified immune cell composition and/or unbound anti-fugetactic agent and/or the at least one additional anti-cancer agent are administered by direct injection into the tumor. In one embodiment, the modified immune cell composition and/or unbound anti-fugetactic agent and/or anti-cancer agent is administered directly into the tumor or into a blood vessel feeding the tumor (e.g., via microcatheter injection into the blood vessels in, near, or feeding into the tumor). In one embodiment, the modified immune cell composition and/or unbound anti-fugetactic agent and/or anti-cancer agent is administered systemically. In a further embodiment, the modified immune cell composition and/or unbound anti-fugetactic agent and/or anti-cancer agent is administered by microcatheter, or an implanted device, and an implanted dosage form. In one embodiment, the modified immune cell composition and/or unbound anti-fugetactic agent is administered subcutaneously. In one embodiment, the modified immune cell composition and/or unbound anti-fugetactic agent is administered intradermally.

In one embodiment, the modified immune cell composition and/or unbound anti-fugetactic agent is administered in a continuous manner for a defined period. In another embodiment, modified immune cell composition and/or unbound anti-fugetactic agent is administered in a pulsatile manner. For example, the modified immune cell composition and/or unbound anti-fugetactic agent may be administered intermittently over a period of time.

In addition, important embodiments of the invention, particularly with regard to administration of unbound anti-fugetactic agent, include pump-based hardware delivery systems, some of which are adapted for implantation. Such implantable pumps include controlled-release microchips. A preferred controlled-release microchip is described in Santini, J T Jr. et al., Nature, 1999, 397:335-338, the contents of which are expressly incorporated herein by reference.

It is to be appreciated that the treatment of tumors or cancers with an effective amount of a modified immune cell composition according to the present disclosure (with or without administration of unbound anti-fugetactic agent) for a period of time sufficient to attenuate the fugetactic activity of the chemokine restores immune defenses against tumors, and may also allow anti-cancer agents (e.g., chemotherapeutic agents, radiotherapeutic agents, immunotherapeutic agents, and the like) to better access the tumor or cancer in order to reduce or eradicate the tumor or cancer. Without being bound by theory, it is believed that co-administration of the modified immune cell compositions as described herein and anti-cancer agents will lead to a synergistic response in a patient with a tumor or cancer, such that the patient has a better outcome than with either therapy alone. Anti-cancer agents include, without limitation, known cancer therapies, e.g. chemotherapy, radiotherapy, immunotherapy and/or vaccine therapy.

The anti-cancer agent may be administered by any appropriate method. Dosage, treatment protocol, and routes of administration for anti-cancer agents, including chemotherapeutic agents, radiotherapeutic agents, immunotherapeutic agents, and anti-cancer vaccines, are known in the art and/or within the ability of a skilled clinician to determine, based on the type of treatment, type of cancer, etc.

In one aspect of the invention, the modified immune cell composition and/or unbound anti-fugetactic agent and/or the anti-cancer agent(s) are administered sequentially. That is, the modified immune cell composition and/or unbound anti-fugetactic agent is administered for a period of time sufficient to allow targeting and/or penetration of the tumor or cancer cells by the modified immune cells, and the anti-cancer agent is subsequently administered.

In one aspect of the invention, the anti-cancer agent is administered after the period of time of administration of modified immune cell composition and/or unbound anti-fugetactic agent. In one embodiment, the anti-cancer agent is administered during a period of time wherein the fugetactic effect of the cancer cells/tumor is attenuated by the modified immune cell composition and/or unbound anti-fugetactic agent. The length of time and modes of administration of the anti-cancer agent will vary, depending on the anti-cancer agent used, type of tumor being treated, condition of the patient, and the like. Determination of such parameters is within the capability of the skilled clinician.

In one embodiment, administration of the modified immune cell composition and/or unbound anti-fugetactic agent and the anti-cancer agent is alternated. In a preferred embodiment, administration of the modified immune cell composition and/or unbound anti-fugetactic agent and the anti-cancer agent is alternated until the condition of the patient improves. Improvement includes, without limitation, reduction in size of the tumor and/or metastases thereof, elimination of the tumor and/or metastases thereof, remission of the cancer, and/or attenuation of at least one symptom of the cancer.

In a preferred embodiment, the modified immune cell composition and/or unbound anti-fugetactic agent and anti-cancer agent(s) are administered sequentially. For example, the modified immune cell composition and/or unbound anti-fugetactic agent may be administered for a period of time sufficient to reduce or attenuate the fugetactic effect of the tumor, e.g. such that the modified immune cell composition and/or unbound anti-fugetactic agent has an anti-fugetactic effect; the anti-cancer agent can then be administered for a period of time during which the fugetactic effect of the tumor is reduced or attenuated. In one embodiment, the modified immune cell composition and/or unbound anti-fugetactic agent and anti-cancer agent are administered sequentially in an alternating manner at least until the condition of the patient improves. Improvement of the condition of the patient includes, without limitation, reduction in tumor size, a reduction in at least one symptom of the cancer, elimination of the tumor and/or metastases thereof, increased survival of the patient, and the like.

### Chemotherapy Agents

In one aspect of the present invention, a modified immune cell composition and/or unbound anti-fugetactic agent is administered in combination with a chemotherapy agent. The chemotherapy agent may be any agent having a therapeutic effect on one or more types of cancer. Many chemotherapy agents are currently known in the art. Types of chemotherapy drugs include, by way of non-limiting example, alkylating agents, antimetabolites, anti-tumor antibiotics, totpoisomerase inhibitors, mitotic inhibitors, corticosteroids, and the like.

Non-limiting examples of chemotherapy drugs include: nitrogen mustards, such as mechlorethamine (nitrogen mustard), chlorambucil, cyclophosphamide (Cytoxan®), ifosfamide, and melphalan); Nitrosoureas, such as streptozocin, carmustine (BCNU), and lomustine; alkyl sulfonates, such as busulfan; Triazines, such as dacarbazine (DTIC) and temozolomide (Temodar®); ethylenimines, such as thiotepa and altretamine (hexamethylmelamine); platinum drugs, such as cisplatin, carboplatin, and oxalaplatin; 5-fluorouracil (5-FU); 6-mercaptopurine (6-MP); Capecitabine (Xeloda®); Cytarabine (Ara-C®); Floxuridine; Fludarabine; Gemcitabine (Gemzar®); Hydroxyurea; Methotrexate; Pemetrexed (Alimta®); anthracyclines, such as Daunorubicin, Doxorubicin (Adriamycin®), Epirubicin, Idarubicin; Actinomycin-D; Bleomycin; Mitomycin-C; Mitoxantrone; Topotecan; Irinotecan (CPT-11); Etoposide (VP-16); Teniposide; Mitoxantrone; Taxanes: paclitaxel (Taxol®) and docetaxel (Taxotere®); Epothilones: ixabepilone (Ixempra®); Vinca alkaloids: vinblastine (Velban®), vincristine (Oncovin®), and vinorelbine (Navelbine®); Estramustine (Emcyt®); Prednisone; Methylprednisolone (Solumedrol®); Dexamethasone (Decadron®); L-asparaginase; bortezomib (Velcade®). Additional chemotherapy agents are listed, for example, in U.S. Patent Application Pub. No. 2008/0300165, which is incorporated herein by reference in its entirety.

Doses and administration protocols for chemotherapy drugs are well-known in the art. The skilled clinician can readily determine the proper dosing regimen to be used, based on factors including the chemotherapy agent(s) administered, type of cancer being treated, stage of the cancer, age and condition of the patient, patient size, location of the tumor, and the like.

### Radiotherapy Agents

In one aspect of the present invention, a modified immune cell composition and/or unbound anti-fugetactic agent is administered in combination with a radiotherapeutic agent. The radiotherapeutic agent may be any such agent having a therapeutic effect on one or more types of cancer. Many radiotherapeutic agents are currently known in the art. Types of radiotherapeutic drugs include, by way of non-limiting example, X-rays, gamma rays, and charged particles. In one embodiment, the radiotherapeutic agent is delivered by a machine outside of the body (external-beam radiation therapy). In a preferred embodiment, the radiotherapeutic agent is placed in the body near the tumor/cancer cells (brachytherapy) or is a systemic radiation therapy.

External-beam radiation therapy may be administered by any means. Non-limiting examples of external-beam radiation therapy include linear accelerator-administered radiation therapy, 3-dimensional conformal radiation therapy (3D-CRT), intensity-modulated radiation therapy (IMRT), image-guided radiation therapy (IGRT), tomotherapy, stereotactic radiosurgery, photon therapy, stereotactic body radiation therapy, proton beam therapy, and electron beam therapy.

Internal radiation therapy (brachytherapy) may be by any technique or agent. Non-limiting examples of internal radiation therapy include any radioactive agents that can be placed proximal to or within the tumor, such as Radium-226 (Ra-226), Cobalt-60 (Co-60), Cesium-137 (Cs-137), cesium-131, Iridium-192 (Ir-192), Gold-198 (Au-198), Iodine-125 (I-125), palladium-103, yttrium-90, etc. Such agents may be administered by seeds, needles, or any other route of administration, and may be temporary or permanent.

Systemic radiation therapy may be by any technique or agent. Non-limiting examples of systemic radiation therapy include radioactive iodine, ibritumomab tiuxetan (Zevalin®), tositumomab and iodine I 131 tositumomab (Bexxar®), samarium-153-lexidronam (Quadramet®), strontium-89 chloride (Metastron®), metaiodobenzylguanidine, lutetium-177, yttrium-90, strontium-89, and the like.

In one embodiment, a radiosensitizing agent is also administered to the patient. Radiosensitizing agents increase the damaging effect of radiation on cancer cells.

Doses and administration protocols for radiotherapy agents are well-known in the art. The skilled clinician can readily determine the proper dosing regimen to be used, based on factors including the agent(s) administered, type of cancer being treated, stage of the cancer, location of the tumor, age and condition of the patient, patient size, and the like.

### Immunotherapy Agents

In one aspect of the present invention, a modified immune cell composition and/or unbound anti-fugetactic agent is administered in combination with an additional immunotherapy agent.

### Natural Killer (NK) Cells

Natural killer (NK) cells are a class of lymphocytes that typically comprise approximately 10% of the lymphocytes in a human. NK cells provide an innate cellular immune response against tumor and infected (target) cells. NK cells, which are characterized as having a CD3-/CD56+ phenotype, display a variety of activating and inhibitory cell surface receptors. NK cell inhibitory receptors predominantly engage with major histocompatibility complex class I (“MHC-I”) proteins on the surface of a normal cell to prevent NK cell activation. The MHC-I molecules define cells as “belonging” to a particular individual. It is thought that NK cells can be activated only by cells on which these “self” MHC-I molecules are missing or defective, such as is often the case for tumor or virus-infected cells.

NK cells are triggered to exert a cytotoxic effect directly against a target cell upon binding or ligation of an activating NK cell receptor to the corresponding ligand on the target cell. The cytotoxic effect is mediated by secretion of a variety of cytokines by the NK cells, which in turn stimulate and recruit other immune system agents to act against the target. Activated NK cells also lyse target cells via the secretion of the enzymes perforin and granzyme, stimulation of apoptosis-initiating receptors, and other mechanisms.

NK cells have been evaluated as an immunotherapeutic agent in the treatment of certain cancers. NK cells used for this purpose may be autologous or non-autologous (i.e., from a donor).

In one embodiment, the NK cells are autologous NK cells. In one embodiment, the NK cells are non-autologous NK cells.

In one embodiment, the NK cells are genetically modified NK cells. NK cells can be genetically modified by insertion of genes or RNA into the cells such that the cells express one or more proteins that are not expressed by wild type NK cells. In one embodiment, the NK cells are genetically modified to express a chimeric antigen receptor (CAR). In a preferred embodiment, the CAR is specific for the cancer being targeted by the method or composition.

Non-limiting examples of modified NK cells can be found, for example, in Glienke, et al. 2015, Advantages and applications of CAR-expressing natural killer cells, Frontiers in Pharmacol. 6, article 21; PCT Patent Pub. Nos. WO 2013154760 and WO 2014055668; each of which is incorporated herein by reference in its entirety.

In some embodiments, the NK cells are an NK cell line. NK cell lines include, without limitation, NK-92, NK-YS, KHYG-1, NKL, NKG, SNK-6, and IMC-1. See, Klingemann et al. Front Immunol. 2016; 7: 91, which is incorporated herein by reference in its entirety.

### NK-92 Cells

The NK-92 cell line was discovered in the blood of a subject suffering from a non-Hodgkins lymphoma. NK-92 cells lack the major inhibitory receptors that are displayed by normal NK cells, but retain a majority of the activating receptors. NK-92 cells are cytotoxic to a significantly broader spectrum of tumor and infected cell types than are NK cells and often exhibit higher levels of cytotoxicity toward these targets. NK-92 cells do not, however, attack normal cells, nor do they elicit an immune rejection response. In addition, NK-92 cells can be readily and stably grown and maintained in continuous cell culture and, thus, can be prepared in large quantities under c-GMP compliant quality control. This combination of characteristics has resulted in NK-92 being entered into presently on-going clinical trials for the treatment of multiple types of cancers.

NK-92 cells may be wild type (i.e., not genetically modified) NK-92 cells or genetically modified NK-92 cells. NK-92 cells can be genetically modified by insertion of genes or RNA into the cells such that the cells express one or more proteins that are not expressed by wild type NK-92 cells. In one embodiment, NK-92 cells are genetically modified to express a chimeric antigen receptor (CAR) on the cell surface. In a preferred embodiment, the CAR is specific for the cancer being targeted by the method or composition. In one embodiment, NK-92 cells are genetically modified to express an Fc receptor on the cell surface. In a preferred embodiment, the NK-92 cell expressing the Fc receptor can mediate antibody-dependent cell-mediated cytotoxicity (ADCC). In one embodiment, the Fc receptor is CD16. In one embodiment, NK-92 cells are genetically modified to express a cytokine (e.g., IL-2).

In one embodiment, the modified NK-92 cell is administered in combination with an antibody specific for the cancer to be treated. In a preferred embodiment, the modified NK-92 cell administered in combination with the antibody is competent to mediate ADCC.

Non-limiting examples of modified NK-92 cells are described, for example, in U.S. Pat. Nos. 7,618,817 and 8,034,332; and U.S. Patent Pub. Nos. 2002/0068044 and 2008/0247990, each of which is incorporated herein by reference in its entirety. Non-limiting examples of CAR-modified NK-92 cells can be found, for example, in Glienke, et al. 2015, Advantages and applications of CAR-expressing natural killer cells, Frontiers in Pharmacol. 6, article 21; which is incorporated herein by reference in its entirety.

### Antibodies

Immunotherapy also refers to treatment with anti-tumor antibodies. That is, antibodies specific for a particular type of cancer (e.g., a cell surface protein expressed by the target cancer cells) can be administered to a patient having cancer. The antibodies may be monoclonal antibodies, polyclonal antibodies, chimeric antibodies, antibody fragments, human antibodies, humanized antibodies, or non-human antibodies (e.g. murine, goat, primate, etc.). The therapeutic antibody may be specific for any tumor-specific or tumor-associated antigen. See, e.g. Scott et al., Cancer Immunity 2012, 12:14, which is incorporated herein by reference in its entirety.

In one embodiment, the immunotherapy agent is an anti-cancer antibody. Non-limiting examples include trastuzumab (Herceptin®), bevacizumab (Avastin®), cetuximab (Erbitux®), panitumumab (Vectibix®), ipilimumab (Yervoy®), rituximab (Rituxan®), alemtuzumab (Campath®), ofatumumab (Arzerra®), gemtuzumab ozogamicin (Mylotarg®), brentuximab vedotin (Adcetris®), ⁹⁰Y-ibritumomab tiuxetan (Zevalin®), and ¹³¹I-tositumomab (Bexxar®).

Additional antibodies are provided in Table 1.

TABLE 1
Anti-cancer antibodies

| | | | Indication first |
|---|---|---|---|
| Proprietary | | | approved or |
| name | Trade name | Target; Format | reviewed |
| Necitumumab | (Pending) | EGFR; Human | Non-small cell |
| | | IgG1 | lung cancer |
| Nivolumab | Opdivo | PD1; Human IgG4 | Melanoma |
| Dinutuximab | (Pending) | GD2; Chimeric | Neuroblastoma |
| | | IgG1 | |
| Blinatumomab | Blincyto | CD19, CD3; | Acute |
| | | Murine bispecific | lymphoblastic |
| | | tandem scFv | leukemia |
| Pembrolizumab | Keytruda | PD1; Humanized | Melanoma |
| | | IgG4 | |
| Ramucirumab | Cyramza | VEGFR2; Human | Gastric cancer |
| | | IgG1 | |
| Obinutuzumab | Gazyva | CD20; Humanized | Chronic |
| | | IgG1; | lymphocytic |
| | | Glycoengineered | leukemia |
| Ado-trastuzumab | Kadcyla | HER2; humanized | Breast cancer |
| emtansine | | IgG1; | |
| | | immunoconjugate | |
| Pertuzumab | Perjeta | HER2; humanized | Breast Cancer |
| | | IgG1 | |
| Brentuximab | Adcetris | CD30; Chimeric | Hodgkin |
| vedotin | | IgG1; | lymphoma, |
| | | immunoconjugate | systemic |
| | | | anaplastic large |
| | | | cell lymphoma |
| Ipilimumab | Yervoy | CTLA-4; Human | Metastatic |
| | | IgG1 | melanoma |
| Ofatumumab | Arzerra | CD20; Human | Chronic |
| | | IgG1 | lymphocytic |
| | | | leukemia |

### Immune Checkpoint Inhibitors

In one embodiment, the immunotherapy agent is a checkpoint inhibitor. Immune checkpoint proteins are made by some types of immune system cells, such as T cells, and some cancer cells. These proteins, which can prevent T cells from killing cancer cells, are targeted by checkpoint inhibitors. Checkpoint inhibitors increase the T cells' ability to kill the cancer cells. Examples of checkpoint proteins found on T cells or cancer cells include PD-1/PD-L1 and CTLA-4/B7-1/B7-2.

In one embodiment, the checkpoint inhibitor is an antibody to a checkpoint protein, e.g., PD-1, PDL-1, or CTLA-4. Checkpoint inhibitor antibodies include, without limitation, BMS-936559, MPDL3280A, MedI-4736, Lambrolizumab, Alemtuzumab, Atezolizumab, Ipilimumab, Nivolumab, Ofatumumab, Pembrolizumab, and Rituximab.

### Cytokines

In one embodiment, the immunotherapy agent is a cytokine. Cytokines stimulate the patient's immune response. Cytokines include interferons and interleukins. In one embodiment, the cytokine is interleukin-2. In one embodiment, the cytokine is interferon-alpha.

### Anti-Cancer Vaccines

In one aspect of the present invention, a modified immune cell composition and/or unbound anti-fugetactic agent is administered in combination with an anti-cancer vaccine (also called cancer vaccine). Anti-cancer vaccines are vaccines that either treat existing cancer or prevent development of a cancer by stimulating an immune reaction to kill the cancer cells. In a preferred embodiment, the anti-cancer vaccine treats existing cancer.

The anti-cancer vaccine may be any such vaccine having a therapeutic effect on one or more types of cancer. Many anti-cancer vaccines are currently known in the art. Such vaccines include, without limitation, dasiprotimut-T, Sipuleucel-T, talimogene laherparepvec, HSPPC-96 complex (Vitespen), L-BLP25, gp100 melanoma vaccine, and any other vaccine that stimulates an immune response to cancer cells when administered to a patient.

### Cancers

Cancers or tumors that can be treated with the modified immune cell compositions and/or unbound anti-fugetactic agent and methods described herein include, but are not limited to: biliary tract cancer; brain cancer, including glioblastomas and medulloblastomas; breast cancer (including inflammatory breast cancer); cervical cancer; choriocarcinoma; colon cancer; endometrial cancer; esophageal cancer, gastric cancer; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer (hepatocarcinoma); lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor. In important embodiments, cancers or tumors escaping immune recognition include glioma, colon carcinoma, colorectal cancer, lymphoid cell-derived leukemia, choriocarcinoma, breast cancer, ovarian cancer, prostate cancer, and melanoma.

In a preferred embodiment, the tumor is a solid tumor. In one embodiment, the tumor is a leukemia. In an especially preferred embodiment, the tumor expresses or over-expresses CXCL12. In one embodiment, tumor expression of CXCL12 can be evaluated prior to administration of a composition as described herein. For example, a patient having a tumor that is determined to express or over-express CXCL12 will be treated using a method and/or composition as described herein.

In one embodiment, the tumor is a brain tumor. It is contemplated that a brain tumor, e.g., an inoperable brain tumor, can be injected with a composition described herein. In one embodiment, an anti-fugetactic agent is administered directly to a brain tumor via a catheter into a blood vessel within or proximal to the brain tumor. Further discussion of catheter or microcatheter administration is described below.

### Pharmaceutical Compositions

The present invention also provides pharmaceutical compositions comprising an effective amount of the modified immune cell compositions of the present invention, with or without unbound anti-fugetactic agent, and one or more pharmaceutically acceptable excipients. For preparing pharmaceutical compositions containing modified immune cell compositions of the present invention, inert and pharmaceutically acceptable excipients or carriers are used. Liquid pharmaceutical compositions include, for example, solutions, suspensions, and emulsions suitable for intradermal, subcutaneous, parenteral, or intravenous administration. Sterile water solutions of the modified immune cell compositions or sterile solutions of the modified immune cell compositions in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

The pharmaceutical compositions containing modified immune cell compositions can be administered for prophylactic and/or therapeutic treatments. In therapeutic applications, compositions are administered to a patient already suffering from a condition that may be exacerbated by the proliferation of tumor or cancer cells in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient. The appropriate dose may be administered in daily, weekly, biweekly, or monthly intervals. Single or multiple administrations of the compositions can be carried out with dose levels and pattern being selected by the treating physician. In any event, the pharmaceutical formulations should provide a quantity of the modified immune cell compositions of this invention sufficient to provide the desired anti-fugetactic properties when administered to the patient, and to effectively inhibit tumor cell growth, proliferation, or survival in the patient for therapeutic purposes.

Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990). The pharmaceutical compositions of the present invention can be administered by various routes, e.g., subcutaneous, intradermal, transdermal, intramuscular, intravenous, or intraperitoneal.

### Methods of Treatment

In one aspect of this invention is provided a method for treating cancer in a patient in need thereof by administration of a modified immune cell composition as described herein. In one embodiment, the modified immune cell composition is administered in combination with at least one additional anti-cancer agent. In one embodiment, the modified immune cell composition is administered in combination with unbound anti-fugetactic agent.

In one aspect, this invention relates to a method for killing a cancer cell expressing an amount of a chemokine sufficient to produce a fugetactic effect, which method comprises a) contacting said cell with an effective amount of a modified immune cell composition as described herein for a sufficient period of time so as to allow the immune cells to overcome the fugetactic effect, e.g. to target the cancer cell. In one embodiment, the method further comprises b) contacting said cell with at least one anti-cancer agent. In one embodiment, the method further comprises repeating a) and/or b) as necessary to kill said cell.

In one aspect, this invention relates to a method for treating a tumor in a mammal, said tumor expressing an amount of a chemokine sufficient to produce a fugetactic effect, which method comprises a) administering to said mammal an effective amount of a modified immune cell composition as described herein for a sufficient period of time so as to allow the immune cells to overcome the fugetactic effect, e.g. to target and/or penetrate the tumor. In one embodiment, the method further comprises a′) administering to said mammal an effective amount of an unbound anti-fugetactic agent for a sufficient period of time so as to attenuate said fugetactic effect, where a′) may be performed before, with, or after a). In one embodiment, the method further comprises b) administering to said mammal at least one anti-cancer agent. In one embodiment, steps a) and/or b) are repeated as necessary to provide an improvement in the condition of the mammal.

In one embodiment, the anti-cancer agent is administered after the period of time of administration of the modified immune cell composition and/or unbound anti-fugetactic agent. In one embodiment, the anti-cancer agent is administered during a period of time when the fugetactic effect is attenuated.

In one embodiment, the chemokine is CXCL12. In one embodiment, the cancer cell is a solid tumor cell. In one embodiment, the cancer cell is a leukemia cell. In one embodiment, the anti-cancer agent is administered within about 3 days of completion of contacting the cell with the anti-fugetactic agent. In one embodiment, the anti-cancer agent is administered within about 1 day of completion of contacting the cell with the anti-fugetactic agent.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1: Determination of the Anti-Fugetactic Versus Fugetactic Amount of AMD3100

Freshly prepared and purified human CD3⁺ T cells were prepared from healthy donor peripheral blood. 20,000 T cells were loaded into the upper chamber of the Transwell in control, chemotactic or fugetactic settings with AMD3100 at concentrations between 0.1 μM and 10 μM. Migrated cells were counted in the lower chamber and migration quantitated as previously described. Vianello et al. The Journal of Immunology, 2006, 176: 2902-2914; Righi et al., Cancer Res.; 71(16); 5522-34, each of which is incorporated herein in its entirety.

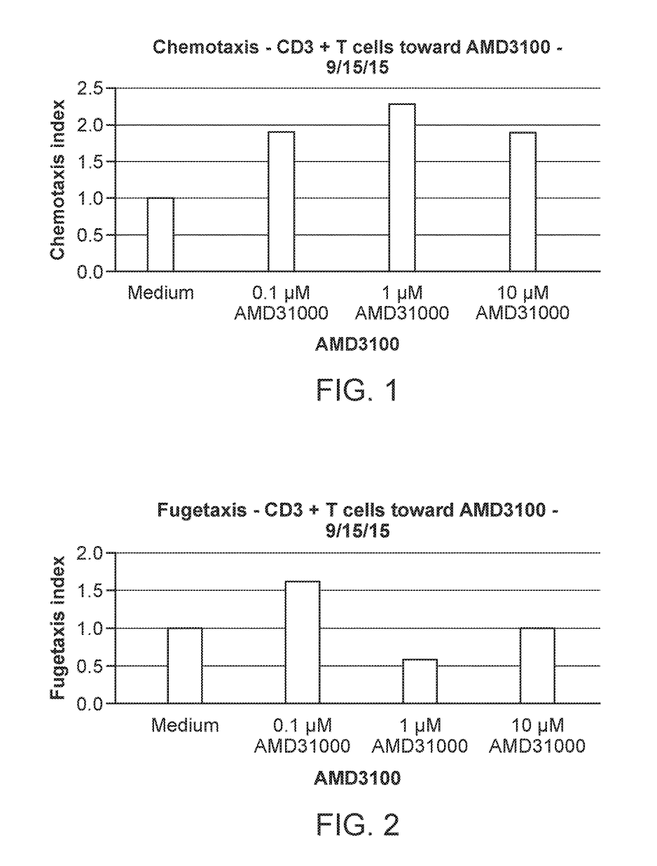

Clear evidence of binary or bimodal chemotactic (FIG. 1; CI 2.3 at 1 μM) and fugetactic (FIG. 2; CI=1.6 at 0.1 μM) responses of human CD3+ T cells to AMD3100 (where a CI or chemotactic index of 1.0 is the control) was observed. All wells were run in triplicate.

### Example 2: Determination of the Local Anti-Fugetactic Amount of AMD3100

For quantitative transmigration assays, purified human CD3⁺ T cells (approximately 2×10⁴ cells) are added to the upper chamber of a Transwell® insert in each well, to a total volume of 150 μl of Iscove's modified medium. Tumor cells isolated from a mammalian tumor in DMEM containing 0.5% FCS, are added in the lower, upper, or both lower and upper chambers of the Transwell to generate a standard “checkerboard” analysis of cell migration, including measurements of chemotaxis, fugetaxis, and chemokinesis.

To determine the anti-fugetactic concentration of AMD3100, the T cells are incubated with 0.01 μM to 10 mM AMD3100 prior to addition to the chamber.

Cells are harvested from the lower chamber after 3 h, and cell counts are performed using a hemocytometer.

It is expected that T cells that are pre-incubated with a concentration of AMD3100 will exhibit a bimodal effect, with anti-fugetactic effects observed at lower concentrations and fugetactic effects at higher concentrations.

### Example 3: Treatment of a Tumor with Modified T Cells and an Anti-Fugetactic Agent

T cells are isolated from a 65 year old patient with glioblastoma and expanded in vitro to provide a T cell population. The T cell population is then mixed and incubated with AMD3100. The patient receives 1.6×10⁷ modified T cells/AMD3100 composition via direct infusion into the tumor. It is contemplated that treatment with modified T cells and AMD3100 will have a synergistic effect, such that the co-treatment results in decrease in tumor size.

1. An ex vivo immune cell population comprising modified human immune cells, said immune cell population having an anti-fugetactic agent bound to individual immune cells through at least one receptor on the cell surface, wherein said immune cell population exhibits overall anti-fugetactic properties relative to a cancer when delivered to a patient in vivo.

2. The cell population of claim 1, wherein said anti-fugetactic agent is selected from the group consisting of AMD3100 or derivative thereof, KRH-1636, T-20, T-22, T-140, TE-14011, T-14012, TN14003, TAK-779, AK602, SCH-351125, Tannic acid, NSC 651016, thalidomide, GF 10923 OX, an antibody that interferes with dimerization of a fugetactic chemokine, and an antibody that interferes with dimerization of a receptor for a fugetactic chemokine.

3. The cell population of claim 1, wherein said anti-fugetactic agent is AMD3100.

4. The cell population of claim 1, wherein the immune cells are T-cells.

5. The cell population of claim 4, wherein the T-cells are selected from the group consisting of allogenic T-cells, autologous T-cells, and immortalized T-cells.

6. The cell population of claim 4, wherein the T-cells are further modified to express a chimeric antigen receptor.

7. A composition comprising the ex vivo immune cell population of claim 1.

8. The composition of claim 7, wherein said anti-fugetactic agent is selected from the group consisting of AMD3100 or derivative thereof, KRH-1636, T-20, T-22, T-140, TE-14011, T-14012, TN14003, TAK-779, AK602, SCH-351125, Tannic acid, NSC 651016, thalidomide, GF 10923 OX, an antibody that interferes with dimerization of a fugetactic chemokine, and an antibody that interferes with dimerization of a receptor for a fugetactic chemokine.

9. The composition of claim 7, wherein said anti-fugetactic agent is AMD3100.

10. The composition of claim 7, wherein the immune cells are T-cells.

11. The composition of claim 10, wherein the T-cells are selected from the group consisting of allogenic T-cells, autologous T-cells, and immortalized T-cells.

12. The composition of claim 10, wherein the T-cells are further modified to express a chimeric antigen receptor.

13. The composition of claim 7, further comprising a pharmaceutically acceptable excipient.

14. The composition of claim 7, further comprising anti-fugetactic agent that is not associated with the immune cells.

15. A method of enhancing tumor penetration of immune cells in a patient having a cancer exhibiting a fugetactic effect, the method comprising administering to the patient an effective amount of the cell population of claim 1.

16. The method of claim 15, further comprising systemically administering a therapeutically effective amount of the anti-fugetactic agent to the patient.

17-19. (canceled)

20. A method of treating a patient having a cancer having a fugetactic effect, the method comprising:
a) extracting autologous immune cells from the patient;
b) modifying the immune cells by contacting the immune cells with an anti-fugetactic agent to provide modified immune cells; and
c) administering the modified immune cells to the patient so as to treat the cancer.

21. The method of claim 20, further comprising systemically administering a therapeutically effective amount of the anti-fugetactic agent to the patient.

22-23. (canceled)

24. The method of claim 20, further comprising modifying the immune cells to express a chimeric antigen receptor that is specific for the cancer.

25. The method of claim 20, wherein the immune cells are T-cells.

26. A method for making a modified immune cell composition having overall anti-fugetactic properties, said method comprising (a) providing immune cells having CXCR4 receptors, and (b) contacting the immune cell population with an anti-fugetactic agent to provide a modified immune cell population.

27. The method of claim 26, wherein said anti-fugetactic agent is selected from the group consisting of AMD3100 or derivative thereof, KRH-1636, T-20, T-22, T-140, TE-1401 1, T-14012, TN14003, TAK-779, AK602, SCH-351 125, Tannic acid, NSC 651016, thalidomide, GF 10923 OX, an antibody that interferes with dimerization of a fugetactic chemokine, and an antibody that interferes with dimerization of a receptor for a fugetactic chemokine.

28. The method of claim 27, wherein said anti-fugetactic agent is AMD3100.

29. The method of claim 26, wherein said immune cells are T-cells.

30. The method of claim 26, wherein providing the T-cells includes extracting autologous immune cells having CXCR4 receptors from a patient having cancer to provide an immune cell population.

31. The method of claim 26, wherein the immune cells are contacted with said anti-fugetactic agent and stored for the subsequent administration to a patient.

32. The method of claim 26, wherein the immune cells are contacted with the anti-fugetactic agent immediately prior to administration of the modified immune cell population to a patient.

33-43. (canceled)
